SEQUENCE LISTING (1 sequence)

SEQ ID NO: 1 - protein, 275 aa, Bacillus arsenicus
AQTTPWGITHIKADQVHATGNTGSGVKVAILDTGIDASHADLNVRGGASFVAGEPNALTDGNSHGTHVAGTVAALNNTTGVLGVAYSADLYAVKVLDSSGSGTYSGIIQGIEWAVANNMDVINMSLGGSSGSTALQQACDNAYNSGVLVVAAAGNSGTRGKQNTIGYPAKYASVMAVGAVDSSNNRASFSSVGSELEVMAPGVNILSTVPGNGYDSYNGTSMASPHVAGAAALIMAGNPGLSNVQVRQKLVNTAKPLGDSFYYGKGVIDVYAATR

(19) United States
(12) Patent Application Publication
(10) Pub. No.: US 2026/0209734 A1
(43) Pub. Date: Jul. 23, 2026

(54) DETERGENTS AND CLEANING AGENTS CONTAINING PROTEASE

(71) Applicant: Henkel AG & Co. KGaA, Düsseldorf (DE)

(72) Inventors: Christian Degering, Erkrath (DE); Susanne Wieland, Zons/Dormagen (DE); Shohana Islam, Dusselforf (DE); Claudia Lindner, Solingen (DE); Petra Siegert, Haan (DE); Fabian Falkenberg, Neuss (DE); Johannes Bongaerts, Dormagen (DE)

(21) Appl. No.: 19/135,011

(22) PCT Filed: Nov. 16, 2023
(86) PCT No.: PCT/EP2023/082000

§ 371 Date: Jun. 2, 2025

(30) Foreign Application Priority Data
Dec. 13, 2022 (DE) 10 2022 213 537.7

(51) Int. Cl. C12N 9/52; C11D 3/386

(52) U.S. Cl. CPC C12N 9/52; C11D 3/38618; C11D 2111/12; C11D 2111/14; C12Y 304/00

(57) ABSTRACT

The invention is in the field of enzyme technology. The invention relates to proteases from Fictibacillus arsenicus that are in particular suitable for use in washing and cleaning agents, to all sufficiently similar proteases having a correspondingly similar sequence to SEQ ID NO:1, and to nucleic acids coding for said proteases. The invention further relates to the production of said proteases, to a method for using said proteases, to their use as such, and to agents containing said proteases, in particular washing and cleaning agents.

The invention is in the field of enzyme technology. The invention relates to proteases that are in particular suitable for use in washing and cleaning agents, in particular in liquid washing and cleaning agents, to all sufficiently similar proteases having a correspondingly similar sequence to SEQ ID NO:1, and to nucleic acids coding for said proteases. The invention further relates to the production of said proteases, to a method for using said proteases, to their use as such, and to agents containing said proteases, in particular washing and cleaning agents, in particular liquid washing and cleaning agents.

Proteases are among the technically most important enzymes. For washing and cleaning agents, they are the longest established enzymes and are contained in virtually all modern, high-performance washing and cleaning agents. They cause the degradation of protein-containing stains on the articles to be cleaned. In turn, proteases of the subtilisin type (subtilases, subtilopeptidases, EC 3.4.21.62) are particularly important, which proteases are serine proteases due to the catalytically active amino acids. They act as unspecific endopeptidases and hydrolyze any acid amide bonds within peptides or proteins. Their optimum pH is usually in the distinctly alkaline range. The article “Subtilases: Subtilisin-like Proteases” by R. Siezen, pages 75-95 in “Subtilisin enzymes,” published by R. Bott and C. Betzel, New York, 1996, gives an overview of this family, for example. Subtilases are naturally formed by microorganisms. In particular, the subtilisins formed and secreted by the Bacillus species are the most significant group of subtilases.

Examples of subtilisin-type proteases that are preferably used in washing and cleaning agents are the subtilisins BPN′ and Carlsberg, protease PB92, subtilisins 147 and 309, the alkaline protease from Bacillus lentus, in particular Bacillus lentus DSM 5483, subtilisin DY and the enzymes thermitase, proteinase K and proteases TW3 and TW7, which are to be classified as subtilases, but no longer as subtilisins in the narrower sense, as well as variants of said proteases that have an amino acid sequence which is altered compared to the starting protease. Proteases are altered in a targeted or random manner by methods known from the prior art and are thus optimized for use in washing and cleaning agents, for example. This includes point, deletion or insertion mutagenesis, or fusion with other proteins or protein parts. Thus, appropriately optimized variants are known for most proteases known from the prior art.

In general, only selected proteases are suitable for use in liquid, surfactant-containing preparations in any case. Many proteases do not exhibit sufficient catalytic performance in such preparations or they are not sufficiently stable. For the use of proteases in cleaning agents, therefore, a high catalytic activity and stability under conditions as they are during a wash cycle is particularly desirable.

Consequently, protease and surfactant-containing liquid formulations from the prior art are disadvantageous in that the proteases contained, under standard washing conditions (e.g., in a temperature range of from approximately 20° C. to approximately 40° C.), do not have satisfactory proteolytic activity and/or are not sufficiently storage-stable and the formulations therefore do not exhibit optimal cleaning performance on protease-sensitive stains. Protease-sensitive stains are preferably selected from the group consisting of blood, egg (yolk), milk and other protein-containing stains. Tough stains are also usually dealt with in typical washing and cleaning agents by bleaching agents and/or further cleaning-active substances in the washing and cleaning agent, such as phosphonates and/or phosphates. However, the difficulties in removing tough stains and soiling are particularly greater when formulating liquid washing and cleaning agents or formulating washing agents for colored textiles, since these typically do not contain any bleaching agents. The desire for sustainability and concerns regarding the environmental compatibility of phosphates and phosphonates in washing and cleaning agents mean that washing and cleaning agents are increasingly being developed which contain little (less) or no phosphate-containing and/or phosphonate-containing compounds.

There is therefore a need to improve the cleaning performance of washing and cleaning agents, in particular liquid washing and cleaning agents, in particular those without bleaching agents and/or without phosphonates and/or without phosphates, in particular with regard to the cleaning performance on protease-sensitive stains, in particular in a temperature range of about 20° C. to about 40° C.

Surprisingly, it has been found that a previously unknown protease from Fictibacillus arsenicus, or proteases that are sufficiently similar thereto (in relation to the sequence identity), is particularly suitable for use in washing or cleaning agents, in particular liquid washing or cleaning agents, since it removes a broad spectrum of protease-sensitive stains under standard washing conditions.

One subject of the invention is therefore a protease comprising an amino acid sequence which has at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to the amino acid sequence given in SEQ ID NO:1 over its entire length, or variants thereof.

The variants according to the invention are characterized in that they are obtained from a protease as a starting molecule by single or multiple conservative amino acid substitution, said protease comprising an amino acid sequence having at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to the amino acid sequence given in SEQ ID NO: 1 over its entire length, or variants thereof. Alternatively or additionally, the variants according to the invention are characterized in that they are obtained from a protease, which comprises an amino acid sequence having at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to the amino acid sequence given in SEQ ID NO:1 over its entire length, as the starting molecule by fragmentation or deletion, insertion or substitution mutagenesis, and comprises an amino acid sequence which matches the starting molecule over a length of at least 190, 200, 210, 220, 230, 240, 250, 260, 270, 271, 272, 273, 274 or 275 contiguous amino acids.

A further subject of the invention is a method for producing a protease, comprising providing a starting protease having at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to the amino acid sequence given in SEQ ID NO: 1 over its entire length, or variants of the starting protease, wherein the variants are as defined above.

A protease within the meaning of the present patent application therefore comprises both the protease as such and a protease produced using a method according to the invention. All statements regarding the protease therefore relate both to the protease as such and to the proteases produced by means of corresponding methods, and also to the corresponding methods, in particular methods for producing the protease.

Further aspects of the invention relate to the nucleic acids coding for these proteases, to non-human host cells containing proteases or nucleic acids according to the invention, and also to agents comprising proteases according to the invention, in particular washing and cleaning agents, in particular liquid washing and cleaning agents, to washing and cleaning methods, and to uses of the proteases according to the invention in washing or cleaning agents for removing protease-sensitive stains which are preferably selected from the group consisting of blood, egg (yolk), milk and other protein-containing stains.

These and other aspects, features and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate but not to limit the invention and that, in particular, the invention is not limited to these examples.

Unless indicated otherwise, all percentages are indicated in terms of weight percent (wt. %).

Numerical ranges that are indicated in the format “from x to y” also include the stated values. If several preferred numerical ranges are specified in this format, it is readily understood that any ranges resulting from the combination of the various endpoints are also included.

“At least one,” as used herein, means one or more, i.e., 1, 2, 3, 4, 5, 6, 7, 8, 9, 10, 11, 12, 13, 14 or more.

The term “washing and cleaning agent” or “washing or cleaning agent,” as used herein, is synonymous with the term “agent” and denotes a composition for cleaning textiles and/or hard surfaces, in particular dishes, as explained in the description.

“Approximately,” “about,” or “roughly,” as used herein in reference to a numerical value, refers to the corresponding numerical value ±10%, preferably ±5%.

“Substantially free of” means that the composition or the agent contains less than 2 wt. %, preferably less than 1 wt. %, more preferably less than 0.5 wt. %, and particularly preferably less than 0.1 wt. %, of the corresponding substance, relative to the total weight of the composition/agent.

“Liquid,” as used herein, includes liquids and gels as well as pasty compositions. It is preferred that the liquid compositions be flowable and pourable at room temperature, but it is also possible for them to have a limit of liquidity.

A substance, e.g., a composition or an agent, is solid according to the definition of the invention if it is in a solid state of matter at 25° C. and 1013 mbar.

A substance, e.g., a composition or an agent, is liquid according to the definition of the invention if it is in a liquid state of matter at 25° C. and 1013 mbar. Liquid also includes gel form.

“Variant,” as used herein, refers to naturally or artificially generated variations of a native protease which has an amino acid sequence which is modified from the reference form.

The present invention is based on the surprising finding by the inventors that a protease according to the invention from Fictibacillus, in particular Fictibacillus arsenicus, which has an amino acid sequence which is at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to the amino acid sequence given in SEQ ID NO:1, effects the removal of protease-sensitive stains which are preferably selected from the group consisting of blood, egg (yolk), milk and other protein-containing stains under standard washing conditions. This is surprising in particular because protease, in particular protease from Fictibacillus, in particular Fictibacillus arsenicus, has not previously been described for use in washing or cleaning agents.

In preferred embodiments, the protease according to the invention is a protease which has proteolytic activity and comprises an amino acid sequence which is at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to the amino acid sequence given in SEQ ID NO:1 over its entire length. Protease variants according to the invention are obtained from a protease having the indicated sequence identity as the starting molecule by single or multiple conservative amino acid substitution. Alternatively or additionally, the variants used are obtained from a protease having the indicated sequence identity as the starting molecule by means of fragmentation or deletion, insertion or substitution mutagenesis and comprise an amino acid sequence which matches the starting molecule over a length of at least 190, 200, 210, 220, 230, 240, 250, 260, 270, 271, 272, 273, 274 or 275 contiguous amino acids.

In preferred embodiments, the use of proteases according to the invention in a washing or cleaning agent, preferably a liquid washing or cleaning agent, leads to an improved cleaning performance of these agents on at least one and increasingly preferably two, three, four, five or six protease-sensitive stain(s), which is/are preferably selected from the group consisting of blood, egg (yolk), milk and other protein-containing stains. Proteases according to the invention consequently enable improved removal of at least one, preferably of multiple, protease-sensitive stain(s) on textiles and/or hard surfaces, for example dishes. Typical protease-sensitive stains include for example blood, egg (yolk), milk and other protein-containing stains. An improvement in cleaning performance according to the invention, in particular in proteolytic cleaning performance, is present when the protease exhibits an improved cleaning performance relative to an agent without protease, and/or improved cleaning performance relative to a reference protease, as described herein, on at least one and increasingly preferably on two, three, four, five or six protease-sensitive stains(s), which is/are preferably selected from the group consisting of blood, egg (yolk), milk and other protein-containing stains.

In preferred embodiments, the protease according to the invention is a protease which has proteolytic activity and comprises an amino acid sequence which is at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to the amino acid sequence given in SEQ ID NO: 1 over its entire length, wherein the protease causes improved cleaning performance of a washing or cleaning agent containing a protease according to the invention, relative to an agent without protease and/or relative to an agent containing a reference protease, on at least one and increasingly preferably on two, three, four, five or six protease-sensitive stain(s), which is/are preferably selected from the group consisting of blood, egg (yolk), milk and other protein-containing stains, wherein the cleaning performance is determined as described in example 2.

Such performance-enhanced washing results on protease-sensitive stains can be achieved in different temperature ranges, for example in a range from about 0° C. to about 100° C., preferably about 20° C. to about 60° C., in particular about 20° C. to about 40° C.

The proteases according to the invention have higher stability in washing or cleaning agents, for example with respect to surfactants and/or bleaching agents and/or chelators, and/or with respect to temperature influences, in particular with respect to high temperatures of, for example, between about 50° C. and about 65° C., in particular about 60° C., and/or with respect to acidic or basic conditions and/or with respect to changes in pH and/or with respect to denaturing or oxidizing agents and/or with respect to (auto) proteolytic degradation and/or with respect to a change in redox ratios. Performance-enhanced protease variants are therefore provided by particularly preferred embodiments of the invention. Such advantageous embodiments of proteases according to the invention consequently enable improved washing results on protease-sensitive stains, which are preferably selected from the group consisting of blood, egg (yolk), milk and other protein-containing stains.

Cleaning performance within the scope of the invention shall be understood to mean the lightening performance on one or multiple stains, in particular on laundry or dishes. Within the scope of the invention, both the washing or cleaning agent, which comprises the protease, or the washing or cleaning liquor formed by said agent, and the protease itself have a respective cleaning performance. The cleaning performance of the enzyme thus contributes to the cleaning performance of the agent, or of the washing or cleaning liquor formed by the agent. The cleaning performance is preferably ascertained as described hereafter.

Washing liquor is understood to mean the ready-to-use solution which contains the washing or cleaning agent and acts on the textiles or fabric or hard surfaces, in particular dishes, and thus comes into contact with the stains present on the textiles or fabrics or hard surfaces. The washing liquor is usually created when the washing or cleaning process begins and the washing or cleaning agent is diluted with water, for example in a dishwasher, a washing machine or in another suitable container.

The proteases according to the invention exhibit enzymatic activity, i.e. they are capable of hydrolyzing peptides and proteins, in particular in a washing or cleaning agent. A protease according to the invention is therefore an enzyme that catalyzes the hydrolysis of amide/peptide bonds in protein/peptide substrates and is thereby capable of cleaving proteins or peptides. Advantageously, the washing and cleaning agent according to the invention has improved cleaning performance, in particular in removing protease-sensitive stains, which are preferably selected from the group consisting of blood, egg (yolk), milk and other protein-containing stains. The washing and cleaning agent according to the invention is particularly preferably suitable for removing protease-sensitive stains, which are selected from the group consisting of blood, egg (yolk), milk and other protein-containing stains.

Furthermore, a protease according to the invention is preferably a mature protease, i.e. the catalytically active molecule without a signal peptide/signal peptides and/or a propeptide/propeptides. Unless otherwise stated, the sequences indicated also refer to mature (processed) enzymes in each case.

In various embodiments of the invention, the protease is a free enzyme. This means that the protease can act directly with all components of an agent and, if the agent is a liquid agent, the protease is directly in contact with the solvent of the agent (e.g., water). In other embodiments, an agent may contain proteases that form an interaction complex with other molecules or that contain a “coating”. In this case, one or more protease molecule(s) can be separated from the other constituents of the agent by a structure surrounding them. Such a separating structure can arise due to, but is not limited to, vesicles, such as a micelle or a liposome. However, the surrounding structure may also be a virus particle, a bacterial cell or a eukaryotic cell. In various embodiments, an agent may for example contain cells of Bacillus pumilus or Bacillus subtilis or other expression strains, which express the protease according to the invention, or contain cell culture supernatants of such cells.

The identity of nucleic acid or amino acid sequences is determined by a sequence comparison. This sequence comparison is based on the BLAST algorithm established and commonly used in the prior art (cf. e.g., Altschul et al. (1990) “Basic local alignment search tool”, J. Mol. Biol., 215:403-410, and Altschul et al. (1997): “Gapped BLAST and PSI-BLAST: a new generation of protein database search programs”, Nucleic Acids Res., 25:3389-3402) and occurs in principle by similar sequences of nucleotides or amino acids in the nucleic acid or amino acid sequences being assigned to one another. A tabular assignment of the relevant positions is referred to as an alignment. A further algorithm available in the prior art is the FASTA algorithm. Sequence comparisons (alignments), in particular multiple sequence comparisons, are created using computer programs. The Clustal series (cf. e.g., Chenna et al. (2003): “Multiple sequence alignment with the Clustal series of programs”, Nucleic Acid Res. 31:3497-3500), T-Coffee (cf. e.g., Notredame et al. (2000): “T-Coffee: A novel method for multiple sequence alignments”, J. Mol. Biol., 302:205-217) or programs based on these programs or algorithms, for example, are frequently used. Also possible are sequence comparisons (alignments) using the computer program Vector NTI® Suite 10.3 (Invitrogen Corporation, 1600 Faraday Avenue, Carlsbad, California, USA) with the specified standard parameters, the AlignX module of which for the sequence comparisons is based on ClustalW, or Clone Manager 10 (use of the scoring matrix BLOSUM 62 for sequence alignment at amino acid level). Unless stated otherwise, the sequence identity indicated herein is determined using the BLAST algorithm.

Such a comparison also allows a conclusion to be drawn about the similarity of the compared sequences to one another. It is usually given in percent identity, i.e., the proportion of identical nucleotides or amino acid residues at the same positions or positions corresponding to one another in an alignment. In the case of amino acid sequences, the broader concept of homology takes conserved amino acid exchanges into account, i.e., amino acids having similar chemical activity, because these usually perform similar chemical activities within the protein. Therefore, the similarity of the compared sequences can also be indicated as percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It may therefore be expedient to relate sequence matches only to individual, optionally small, regions. Unless otherwise stated, however, identity or homology information in the present application relates to the entire length of the particular nucleic acid or amino acid sequence indicated.

In the context of the present invention, the indication that an amino acid position corresponds to a numerically designated position in SEQ ID NO:1 therefore means that the corresponding position is associated with the numerically designated position in SEQ ID NO:1 in an alignment as defined above. Furthermore, the assignment of the positions is based on the mature protein. This assignment is also to be used in particular when the amino acid sequence of a protease comprises a higher number of amino acid residues than the protease according to SEQ ID NO:1.

In a further embodiment, proteases according to the invention can have amino acid variations, in particular amino acid substitutions, insertions, or deletions. Such proteases are developed, for example, by targeted genetic alteration, i.e., by means of mutagenesis methods, and optimized for specific use purposes or with regard to specific properties (for example with regard to their catalytic activity, stability, etc.). Furthermore, nucleic acids according to the invention can be introduced into recombination approaches and can thus be used to generate completely new types of enzymes or other polypeptides. The aim is to introduce targeted mutations, such as substitutions, insertions or deletions, into the known molecules in order, for example, to improve the cleaning performance of enzymes according to the invention. For this purpose, in particular the surface charges and/or the isoelectric point of the molecules and thus their interactions with the substrate can be altered. For example, the net charge of the enzymes can be altered in order to influence the substrate binding in particular for use in washing and cleaning agents. Alternatively or additionally, the stability or catalytic activity of the protease can be increased by one or more corresponding mutations and its cleaning performance can thereby be improved. Advantageous properties of individual mutations, e.g., individual substitutions, can complement one another. A protease which has already been optimized with regard to specific properties, for example with regard to its activity and/or its tolerance in relation to the substrate spectrum, can therefore also be developed within the scope of the invention.

For the description of substitutions that relate to exactly one amino acid position (amino acid exchanges), the following convention is applied herein: first, the naturally present amino acid is referred to in the form of the internationally used single-letter code, followed by the associated sequence position and finally the inserted amino acid. Several or alternative exchanges within the same polypeptide chain are separated by slashes. “130D/V” thus means that position 130 has mutated to D or V. In the case of insertions, additional amino acids are named according to the sequence position. In the case of deletions, the missing amino acid is replaced by a symbol, for example a star or a dash, or a Δ is indicated before the corresponding position. For example, P9T describes the substitution of proline at position 9 by threonine, P9TH describes the insertion of histidine following the amino acid threonine at position 9 and P9* or ΔP9 describes the deletion of proline at position 9. This nomenclature is known to a person skilled in the art in the field of enzyme technology.

Another subject of the invention is therefore a protease which is characterized in that it is obtained from a protease as described herein as the starting molecule by single or multiple conservative amino acid substitution. The term “conservative amino acid substitution” means the exchange (substitution) of one amino acid functional group for another amino acid functional group, with this exchange not resulting in a change to the polarity or charge at the position of the exchanged amino acid, e.g., the exchange of a nonpolar amino acid functional group for another nonpolar amino acid functional group. Conservative amino acid substitutions within the scope of the invention include, for example: G=A=S, I=V=L=M, D=E, N=Q, K=R, Y=F, S=T, G=A=I=V=L=M=Y=F=W=P=S=T. Before and or after the conservative amino acid substitution, the protease can comprise an amino acid sequence which is at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to the amino acid sequence given in SEQ ID NO:1 over its entire length.

Alternatively or additionally, the protease is characterized in that it is obtained from a protease according to the invention as the starting molecule by fragmentation or deletion, insertion or substitution mutagenesis and comprises an amino acid sequence which matches the starting molecule over a length of at least 190, 200, 210, 220, 230, 240, 250, 260, 270, 271, 272, 273, 274 or 275 contiguous amino acids. Before and/or after the fragmentation or deletion, insertion or substitution mutagenesis, the protease can comprise an amino acid sequence which is at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to the amino acid sequence given in SEQ ID NO:1 over its entire length.

It is thus possible, for example, to delete individual amino acids at the termini or in the loops of the enzyme without the catalytic activity being lost or reduced as a result. Furthermore, such fragmentation or deletion, insertion or substitution mutagenesis can also be used, for example, to reduce the allergenicity of the enzymes concerned and thus to improve their usability overall. Advantageously, the enzymes retain their catalytic activity even after mutagenesis, i.e., their catalytic activity corresponds at least to that of the starting enzyme, i.e., in a preferred embodiment, the catalytic activity is at least 80%, preferably at least 90%, more preferably at least 100%, of the activity of the starting enzyme. Further substitutions can also demonstrate advantageous effects. Both individual and multiple contiguous amino acids can be replaced with other amino acids.

Further confirmation of the correct assignment of the amino acids to be altered, i.e., in particular their functional correspondence, can be provided by comparative tests, based on which the two positions assigned to one another on the basis of an alignment in the two proteases compared with one another are altered in the same way and observation is carried out to determine whether the enzymatic activity is altered in the same way in the two proteases. If, for example, an amino acid exchange in a specific position of the protease according to SEQ ID NO:1 is accompanied by an alteration of an enzymatic parameter, for example an increase in the KM value, and a corresponding alteration of the enzymatic parameter, for example likewise an increase in the KM value, is observed in a protease variant according to the invention of which the amino acid exchange has been achieved by the same introduced amino acid, this can therefore be considered to be confirmation of the correct assignment.

In particular, according to the invention, fragments of the protease as defined herein are also covered, in particular those according to SEQ ID NO:1 which are truncated at the N-terminus and/or C-terminus such that one or more amino acids of the protease, for example 1, 2, 3, 4, 5, 6, 7, 8, 9 or 10, are no longer present. In various embodiments of the invention, variants of these truncated fragments can also be used which are at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical to the form truncated (in each case) by 1 to 10 N-terminal and/or C-terminal amino acids starting from the amino acid sequence given in SEQ ID NO: 1, over the entire length. For example, proteases comprising an amino acid sequence, which goes beyond the protease comprising an amino acid sequence having at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to the amino acid sequence given in SEQ ID NO:1 over its entire length, or the variants thereof described herein, are also covered by the invention, without the catalytic activity being lost or reduced as a result. Such proteases are preferably those which have additional N-terminal and/or C-terminal amino acids, for example the signal peptide or fragments of the signal peptide, wherein the signal peptide or the fragments of the signal peptide are formed during the production of the protease.

All of these aspects are also applicable to the methods according to the invention for producing a protease.

Accordingly, a method according to the invention further comprises one or more of the following method steps:
(a) providing a starting protease which has at least 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% sequence identity to the amino acid sequence given in SEQ ID NO: 1 over its entire length, or variants thereof;
(b) introducing a single or multiple conservative amino acid substitution;
(c) altering the amino acid sequence by fragmentation or deletion, insertion or substitution mutagenesis such that the protease comprises an amino acid sequence that matches the starting molecule over a length of at least 190, 200, 210, 220, 230, 240, 250, 260, 270, 271, 272, 273, 274 or 275 contiguous amino acids.

All embodiments also apply to the methods according to the invention.

In a further embodiment of the invention, a previously described protease is stabilized, in particular by means of one or more mutations, e.g., substitutions, or by means of coupling to a polymer. An increase in stability during storage and/or during use, for example during the washing process, results in the enzymatic activity lasting longer and thus improves the cleaning performance. In principle, all stabilization options described and/or expedient in the prior art are conceivable. Preference is given to those stabilizations which are achieved via mutations of the enzyme itself because such stabilizations do not require any further working steps after the recovery of the enzyme. Examples of sequence alterations suitable for this purpose are specified above. Further suitable sequence alterations are known from the prior art.

Further possibilities for stabilization are, for example:
altering the binding of metal ions, in particular the calcium binding sites, for example by exchanging one or more of the amino acid(s) involved in the calcium binding for one or more negatively charged amino acids and/or by introducing sequence changes in at least one of the sequences of the two amino acids arginine/glycine;
protecting against the influence of denaturing agents, such as surfactants, by mutations which cause an alteration of the amino acid sequence on or at the surface of the protein;
exchanging amino acids that are close to the N-terminus for those that presumably come into contact with the rest of the molecule via non-covalent interactions and thus contribute to maintaining the globular structure.

Preferred embodiments are those in which the enzyme is stabilized in a plurality of ways because a plurality of stabilizing mutations act additively or synergistically.

In a further embodiment of the invention, the protease is characterized in that its cleaning performance compared to the wild-type enzyme (SEQ ID NO:1) is not significantly reduced; in other words, has at least 70% and increasingly preferably 75%, 80%, 85%, 90% or 95% of the reference washing performance, and more preferably at least 100%, even more preferably at least 110%, particularly preferably at least 120% or more.

The cleaning performance can be determined in a washing system containing a washing agent in a dosage between 2.0 and 8.0 grams per liter of washing liquor and the enzyme. The enzymes to be compared are used in the same concentration (based on active protein). The activity-equivalent use of the relevant enzyme ensures that the respective enzymatic properties, for example the cleaning performance on certain stains, are compared even if the ratio of active substance to total protein (the values of the specific activity) diverges. In general, a low specific activity can be compensated by adding a larger amount of protein. Furthermore, the enzymes to be examined can also be used in the same amount of substance or amount by weight if the enzymes to be examined have a different affinity for the test substrate in an activity test. The expression “same amount of substance” in this context relates to a molar use of the enzymes to be examined. The expression “equal weight” relates to the use of the same weight of the enzymes to be examined.

The concentration of the protease in the washing agent intended for such a washing system is 0.001 to 0.1 wt. %, preferably 0.01 to 0.06 wt. %, relative to active protein.

Washing or cleaning performance is understood to mean the ability of a washing or cleaning agent to partially or completely remove an existing stain, in particular the lightening performance on one or more stains on textiles, in particular cotton textiles, polyester textiles and/or mixed cotton-polyester textiles. Examples of such stains are blood on cotton or chocolate milk/soot on cotton, cocoa on cotton, egg yolk on cotton, milk/soot on cotton or porridge on cotton, etc. Further examples include the aforementioned stains on cotton-polyester blended textiles or polyester-containing textiles or other blended textiles. Within the scope of the invention, both the washing or cleaning agent, which comprises the protease, or the washing or cleaning liquor formed by this agent, and the protease itself have a cleaning performance. The cleaning performance of the protease thus contributes to the cleaning performance of the agent or the washing or cleaning liquor formed by the agent.

Washing or cleaning liquor is understood to mean the ready-to-use solution which contains the washing or cleaning agent and acts on the textiles or hard surfaces, in particular dishes, and thus comes into contact with the stains present on the textiles or hard surfaces. The washing or cleaning liquor is usually created when the washing or cleaning process begins and the washing or cleaning agent is diluted with water, for example in a washing machine or dishwasher or in another suitable container.

A liquid reference washing agent for such a washing system may be composed, for example, as follows (all figures in percent by weight (wt. %)): 4.4% alkyl benzene sulfonic acid, 5.6% further anionic surfactants, 2.4% C₁₂-C₁₈ Na salts of fatty acids (soaps), 4.4% non-ionic surfactants, 0.2% phosphonates, 1.4% citric acid, 0.95% NaOH, 0.01% defoamer, 2% glycerol, 0.08% preservatives, 1% ethanol, and the remainder being demineralized water. The dosage of the liquid washing agent is preferably between 4.5 and 6.0 grams per liter of washing liquor, for example 4.7, 4.9 or 5.9 grams per liter of washing liquor. The washing process preferably takes place in a pH range between pH 7 and pH 10.5, preferably between pH 8 and pH 9.

The cleaning performance is determined with respect to a stain on cotton by measuring the degree of cleaning of the washed textiles. For example, the washing process can take place for 60 minutes at a temperature of approximately 20° C. or approximately 40° C. and the water can have a water hardness between 15.5° dH and 16.5° dH (German hardness). In the context of the invention, the cleaning performance is determined, for example, at 20° C. or 40° C. using a liquid washing agent, for example that specified above, wherein the washing process is preferably carried out for 60 minutes at 600 rpm.

The degree of whiteness, i.e., the lightening of the stains, as a measure of the cleaning performance is determined using optical measuring methods, preferably photometrically. A suitable device for this is, for example, the Minolta CM508d spectrometer. Usually, the devices used for measurement are calibrated beforehand using a white standard, preferably a supplied white standard.

Preferred embodiments of proteases according to the invention achieve such advantageous cleaning performance even at low temperatures, in particular in the temperature ranges between approximately 10° C. and approximately 60° C., preferably between approximately 15° C. and approximately 50° C. and particularly preferably between approximately 20° C. and approximately 40° C.

Methods for determining protease activity are familiar to and routinely used by a person skilled in the art in the field of enzyme technology. For example, such methods are disclosed in Tenside, volume 7 (1970), pp. 125-132. Alternatively, the protease activity can be determined via the release of the chromophore para-nitroaniline (pNA) from the substrate suc-L-Ala-L-Ala-L-Pro-L-Phe-p-nitroanilide (AAPF). The protease cleaves the substrate and releases pNA. The release of the pNA causes an increase in absorbance at 410 nm, the time profile of which is a measure of the enzymatic activity (cf. Del Mar et al., 1979). The measurement is carried out at a temperature of 25° C., a pH of 8.6 and a wavelength of 410 nm. The measurement time is 5 min and the measurement interval is 20 s to 60 s. The protease activity is usually expressed in protease units (PU). Suitable protease activities amount to 2.25, 5 or 10 PE per mL of washing liquor, for example. However, protease activity is not equal to zero.

An alternative test for determining the proteolytic activity of the proteases according to the invention is an optical measurement method, preferably a photometric method. The test suitable for this purpose comprises protease-dependent cleavage of the substrate protein casein. The casein is cleaved by the protease into a plurality of smaller partial products. The entirety of these partial products has an increased absorption at 290 nm with respect to non-cleaved casein, wherein this increased absorption can be determined using a photometer, and thus a conclusion can be drawn regarding the enzymatic activity of the protease.

The protein concentration can be determined using known methods, for example the BCA method (bicinchoninic acid; 2,2′-bichinolyl-4,4′-dicarboxylic acid) or the Biuret method (Gornall at al., J. Biol. Chem. 177 (1948): 751-766). In this regard, the active protein concentration can be determined via titration of the active centers using a suitable irreversible inhibitor and determination of the residual activity (cf. Bender et al., J. Am. Chem. Soc. 88, 24 (1966): 5890-5913).

A further subject of the invention is a protease as described above which is characterized in that it has at least one chemical modification. A protease having such an alteration is referred to as a derivative, i.e., the protease is derivatized. In the context of the present application, derivatives are thus understood to mean proteins whose pure amino acid chain has been chemically modified. Such derivatizations can, for example, be made in vivo by the host cell that expresses the protein. In this regard, couplings of low-molecular-weight compounds, such as lipids or oligosaccharides, are particularly noteworthy. Derivatizations can also be made in vitro, for instance by means of chemical conversion of a side chain of an amino acid or by means of covalent bonding of another compound to the protein. For example, it is possible to couple amines to carboxyl groups of an enzyme in order to alter the isoelectric point. Another such compound can also be a further protein that is bound to a protein according to the invention via bifunctional chemical compounds, for example. Derivatization is likewise understood to mean covalent bonding to a macromolecular carrier or a non-covalent inclusion in suitable macromolecular cage structures. Derivatizations can influence, for example, the substrate specificity or the binding strength to the substrate or bring about temporary blocking of the enzymatic activity if the coupled substance is an inhibitor. This can be expedient for the period of storage, for example. Such modifications may further affect the stability or enzymatic activity. They can also serve to reduce the allergenicity and/or immunogenicity of the protein and thus to increase the skin compatibility thereof, for example. For example, couplings with macromolecular compounds, for example polyethylene glycol, can improve the protein with regard to stability and/or skin compatibility. Derivatives of a protein according to the invention can also be understood in the broadest sense to be preparations of these proteins. A protein can, depending on the recovery, processing or preparation thereof, be combined with various other substances, for example from the culture of the producing microorganisms. A protein can also have been deliberately admixed with other substances, for example to increase its storage stability. Therefore, all preparations of a protein according to the invention are also in accordance with the invention. This is also independent of whether or not it actually exhibits this enzymatic activity in a particular preparation. This is because it may be desirable for it to have no activity or only a small amount of activity during storage and to only exhibit its enzymatic function at the time of use. This can be controlled, for example, via corresponding accompanying substances. In particular, the joint preparation of proteases with specific inhibitors is possible in this regard. In the context of the present invention, of all of the proteases or protease variants and/or derivatives described herein, those for which the storage stability and/or catalytic activity and/or substrate tolerance and/or cleaning performance corresponds to that of the protease according to SEQ ID NO:1 and/or is improved relative to the protease according to SEQ ID NO: 1 are particularly preferred, wherein the catalytic activity and/or cleaning performance is determined as described herein.

The proteases according to the invention and those which can be used in the washing agents according to the invention are obtained from plants, fungi and/or bacteria. The protease comprising SEQ ID NO:1 can be obtained from Fictibacillus arsenicus.

However, the natural production quantities of proteases are often very low. It may therefore be expedient to increase production by expressing protease genes in foreign production hosts. For this purpose, vectors which contain a nucleic acid which codes for a protease according to the invention are generally used.

A further subject matter of invention is a nucleic acid coding for a protease according to the invention, and a vector containing such a nucleic acid, in particular a cloning vector or an expression vector. These can be DNA or RNA molecules. They can be present as a single strand, as a single strand complementary to said single strand or as a double strand. In particular in the case of DNA molecules, the sequences of the two complementary strands must be taken into account in all three possible reading frames. Furthermore, it must be taken into account that different codons, i.e., base triplets, can code for the same amino acids such that a certain amino acid sequence can be coded by a plurality of different nucleic acids. Due to this degeneracy of the genetic code, all of the nucleic acid sequences which can code any of the proteases described above are included in this subject matter of the invention. A person skilled in the art is able to determine these nucleic acid sequences beyond a doubt because, despite the degeneracy of the genetic code, defined amino acids can be assigned to individual codons. Therefore, a person skilled in the art proceeding from said amino acid sequence can easily determine nucleic acids coding for said amino acid sequence. Furthermore, in the case of nucleic acids according to the invention, one or more codons can be replaced by synonymous codons. This aspect relates in particular to the heterologous expression of the enzymes according to the invention. Thus, each organism, for example a host cell of a production strain, has a certain codon usage. “Codon usage” is understood to mean the translation of the genetic code into amino acids by the relevant organism. Bottlenecks can occur in protein biosynthesis if the codons on the nucleic acid in the organism are faced with a comparatively small number of loaded tRNA molecules. Although coding for the same amino acid, this results in a codon being translated less efficiently in the organism than a synonymous codon coding for the same amino acid. Due to the presence of a higher number of tRNA molecules for the synonymous codon, this can be translated more efficiently in the organism. It is possible for a person skilled in the art to use methods which are currently generally known, for example chemical synthesis or polymerase chain reaction (PCR), in conjunction with molecular biology and/or protein-chemical standard methods, to produce the corresponding nucleic acids and even complete genes on the basis of known DNA and/or amino acid sequences. Such methods are known, for example, from Sambrook, J., Fritsch, E. F. and Maniatis, T. 2001. Molecular cloning: a laboratory manual, 3. Edition Cold Spring Laboratory Press.

For the purposes of the present invention, “vectors” are understood to mean elements consisting of nucleic acids that contain a nucleic acid according to the invention as the characteristic nucleic acid region. They are able to establish these as a stable genetic element in a species or cell line over several generations or cell divisions. Vectors are, in particular when used in bacteria, special plasmids, i.e., circular genetic elements. In the context of the present invention, a nucleic acid according to the invention is cloned into a vector. The vectors include, for example, those originating from bacterial plasmids, viruses or bacteriophages, or predominantly synthetic vectors or plasmids with elements of a wide variety of origins. With the other genetic elements present in each case, vectors are able to establish themselves as stable units in the particular host cells over several generations. They can be present extrachromosomally as separate units or can be integrated into a chromosome or chromosomal DNA. Expression vectors comprise nucleic acid sequences that allow them to replicate in the host cells containing them, preferably microorganisms, particularly preferably bacteria, and to express a contained nucleic acid there. The expression is influenced in particular by the promoter or promoters that regulate transcription. In principle, the expression can take place by the natural promoter originally located before the nucleic acid to be expressed, but also by a promoter of the host cell provided on the expression vector or also by a modified or completely different promoter of another organism or another host cell. In the present case, at least one promoter is provided for the expression of a nucleic acid according to the invention and used for the expression thereof. Expression vectors can also be regulatable, for example by changing the cultivation conditions or when a certain cell density of the host cells containing them is reached or by adding certain substances, in particular activators of gene expression. An example of such a substance is the galactose derivative isopropyl β-D-thiogalactopyranoside (IPTG), which is used as an activator of the bacterial lactose operon (lac operon). In contrast to expression vectors, the nucleic acid contained is not expressed in cloning vectors.

The invention further relates to a non-human host cell containing a nucleic acid according to the invention or a vector according to the invention, or containing a protease according to the invention, in particular one that secretes the protease into the medium surrounding the host cell. Preferably, a nucleic acid according to the invention or a vector according to the invention is transformed into a microorganism that then represents a host cell according to the invention. Alternatively, individual components, i.e., nucleic acid parts or fragments of a nucleic acid according to the invention, can also be introduced into a host cell in such a way that the resulting host cell contains a nucleic acid according to the invention or a vector according to the invention. This procedure is particularly suitable when the host cell already contains one or more constituents of a nucleic acid according to the invention or a vector according to the invention and the further constituents are then supplemented accordingly. Methods for transforming cells are established in the prior art and are well known to a person skilled in the art. In principle all cells, i.e., prokaryotic or eukaryotic cells, are suitable as host cells. Host cells which can be managed in a genetically advantageous manner, for example with regard to transformation with the nucleic acid or the vector and its stable establishment, are preferred, for example single-cell fungi or bacteria. Furthermore, preferred host cells are distinguished by good microbiological and biotechnological manageability. This relates, for example, to easy cultivation, high growth rates, low requirements for fermentation media and good production and secretion rates for foreign proteins. Preferred host cells according to the invention secrete the (transgenically) expressed protein into the medium surrounding the host cells. Furthermore, the proteases can be modified by the cells producing them after their production, for example by linking sugar molecules, formylations, aminations, etc. Such post-translational modifications can functionally influence the protease.

Further preferred embodiments are host cells that can be regulated in their activity owing to genetic regulatory elements that are provided, for example, on the vector but can also be present in these cells from the outset. Expression in said cells may be induced, for example, by controlled addition of chemical compounds used as activators, by changing the cultivation conditions or when a particular cell density is reached. This allows economic production of the proteins according to the invention. An example of such a compound is IPTG, as described above.

Prokaryotic or bacterial cells are preferred host cells. Bacteria are characterized by short generation times and low demands on cultivation conditions. This makes it possible to establish cost-effective cultivation methods or production methods. In addition, a person skilled in the art will have a wealth of experience in the case of bacteria in fermentation technology. Gram-negative or gram-positive bacteria can be suitable for a specific production for many different reasons to be determined experimentally in each individual case, such as nutrient sources, product formation rate, time needed, etc. In gram-negative bacteria, such as Escherichia coli, a plurality of proteins are secreted into the periplasmic space, i.e., into the compartment between the two membranes enclosing the cells. This can be advantageous for specific applications. Furthermore, gram-negative bacteria can also be designed such that they discharge the expressed proteins not only into the periplasmic space but into the medium surrounding the bacterium. Gram-positive bacteria, such as, for example, bacilli or actinomycetes or other representatives of Actinomycetes, in contrast do not have an outer membrane, such that secreted proteins are immediately released into the medium surrounding the bacteria, usually the nutrient medium, from which the expressed proteins can be purified. They can be isolated directly from the medium or further processed. In addition, gram-positive bacteria are related or identical to most origin organisms for technically important enzymes and usually themselves form comparable enzymes, such that they have a similar codon usage and their protein synthesis apparatus is naturally aligned accordingly. Host cells according to the invention may be altered in terms of their requirements for culture conditions, have different or additional selection markers, or also express different or additional proteins. In particular, this may also involve those host cells which express a plurality of proteins or enzymes. The present invention can be applied in principle to all microorganisms, in particular to all fermentable microorganisms, particularly preferably to those of the genus Bacillus, and allows proteins according to the invention to be produced using such microorganisms. Such microorganisms then represent host cells for the purposes of the invention. In a further embodiment of the invention, the host cell is characterized in that it is a bacterium, preferably one selected from the group of the genera of Escherichia, Klebsiella, Bacillus, Staphylococcus, Corynebacterium, Arthrobacter, Streptomyces, Stenotrophomonas and Pseudomonas, more preferably one selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

The host cell may also be a eukaryotic cell, however, which is characterized in that it has a cell nucleus. The invention therefore further relates to a host cell that is characterized in that it has a nucleus. In contrast with prokaryotic cells, eukaryotic cells are capable of post-translationally modifying the protein formed. Examples thereof are fungi, such as actinomycetes or yeasts, such as Saccharomyces or Kluyveromyces. This can be particularly advantageous, for example, if the proteins are to undergo specific modifications in connection with their synthesis, which modifications make such systems possible. Modifications carried out by eukaryotic systems, in particular in connection with the protein synthesis, include, for example, the binding of low-molecular-weight compounds, such as membrane anchors or oligosaccharides. Such oligosaccharide modifications can be desirable, for example, to reduce the allergenicity of an expressed protein. Coexpression with the enzymes naturally formed by such cells, such as cellulases, can also be advantageous. Furthermore, for example, thermophilic fungal expression systems can be particularly suitable for expression of temperature-resistant proteins or variants.

The host cells according to the invention are cultured and fermented in the usual manner, for example in discontinuous or continuous systems. In the first case, a suitable nutrient medium is inoculated with the host cells and the product is harvested from the medium after a period to be determined experimentally. Continuous fermentations are characterized by achieving a flow equilibrium in which cells partially die off over a comparatively long period but also grow back and, at the same time, the protein formed can be removed from the medium.

Host cells according to the invention are preferably used to produce proteases according to the invention. The invention therefore further relates to a method for producing a protease, comprising
(a) cultivating a host cell according to the invention, and
(b) isolating the protease from the culture medium or from the host cell.

This subject matter of the invention preferably comprises fermentation processes. Fermentation processes are known per se from the prior art and represent the actual large-scale production step, generally followed by a suitable purification method for the product produced, for example for the proteases according to the invention. All fermentation processes that are based on a corresponding method for producing a protease according to the invention represent embodiments of this subject matter of the invention. Fermentation processes that are characterized in that the fermentation is carried out via a feed strategy are considered in particular. In this case, the media constituents that are consumed by the continuous cultivation are added. As a result, considerable increases can be achieved both in the cell density and in the cell mass or dry mass and/or in particular in the activity of the protease of interest. Furthermore, the fermentation can also be designed such that unwanted metabolic products are filtered out or neutralized by adding buffers or suitable counterions. The prepared protease can be harvested from the fermentation medium. Such a fermentation process is preferred over isolation of the protease from the host cell, i.e., product preparation from the cell mass (dry mass), but requires the provision of suitable host cells or one or more suitable secretion markers or mechanisms and/or transport systems so that the host cells secrete the protease into the fermentation medium. Without secretion, the isolation of the protease from the host cell, i.e., purification thereof from the cell mass, can alternatively take place, for example by means of precipitation with ammonium sulfate or ethanol or by means of chromatographic purification.

All of the above-mentioned aspects can be combined to form methods in order to produce the protease according to the invention.

Another subject of the invention is an agent that is characterized in that it contains a protease according to the invention, as described herein. The agent is preferably a washing or cleaning agent, more preferably a liquid washing or cleaning agent, particularly preferably liquid washing agent.

Particularly preferably, the washing and cleaning agent is substantially free from boron-containing compounds. In preferred embodiments, the protease according to the invention is used in agents or compositions which are substantially free from boron-containing compounds. “Substantially free from boron-containing compounds” in this context means that the agents according to the invention contain less than 2 wt. %, preferably less than 1 wt. %, more preferably less than 0.5 wt. %, and particularly preferably less than 0.1 wt. %, of boron-containing compounds, based on the total weight of the agent. In very particularly preferred embodiments, the washing and cleaning agents according to the invention are free from boron-containing compounds, i.e., in particular they do not contain boric acid and/or phenylboronic acid derivatives.

In preferred embodiments, the protease according to the invention is used in agents or compositions which are substantially free from phosphonate-containing compounds. “Substantially free from phosphonate-containing compounds” in this context means that the corresponding agents or compositions contain less than 2 wt. %, preferably less than 1 wt. %, more preferably less than 0.5 wt. %, and particularly preferably less than 0.1 wt. %, of phosphonate-containing compounds, based on the total weight of the agent/composition. In particularly preferred embodiments, these agents/compositions are free from phosphonate-containing compounds.

In preferred embodiments, the protease according to the invention is used in agents or compositions which are substantially free from phosphate-containing compounds. “Substantially free from phosphate-containing compounds” in this context means that the corresponding agents or compositions contain less than 2 wt. %, preferably less than 1 wt. %, more preferably less than 0.5 wt. %, and particularly preferably less than 0.1 wt. %, phosphate-containing compounds, based on the total weight of the agent/composition. In particularly preferred embodiments, these agents/compositions are free from phosphate-containing compounds.

According to the invention, all conceivable types of washing or cleaning agents are to be understood as washing or cleaning agents, both concentrates and undiluted agents, for use on a commercial scale, in washing machines or for hand washing or cleaning. These include, for example, washing agents for textiles, carpets or natural fibers for which the term washing agent is used. These include, for example, dishwashing detergents for dishwashers (dishwashing detergents) or manual dishwashing detergents or cleaners for hard surfaces such as metal, glass, porcelain, ceramics, tiles, stone, painted surfaces, plastics, wood or leather, for which the term cleaning agent is used, i.e., in addition to manual and mechanical dishwashing detergents, also, for example, scouring agents, glass cleaners, WC toilet scenters, etc. The washing and cleaning agents according to the invention also include auxiliary washing agents which are added to the actual washing agent during manual or automatic textile washing in order to achieve a further effect. Furthermore, in the context of the invention, washing and cleaning agents also include textile pre-treatment agents and post-treatment agents, i.e., those agents with which the item of laundry is brought into contact before the actual washing, for example for dissolving stubborn stains, and also those agents which give the laundry further desirable properties, such as a pleasant feel, crease resistance or a low static charge in a step downstream of the actual textile washing. Inter alia, softeners are included in the latter agents.

The washing or cleaning agents according to the invention, which may be in the form of powdered or granular solids, in compacted or further-compacted particulate form, homogeneous solutions or suspensions, may contain, in addition to a protease according to the invention, all known ingredients conventional in such agents, with preferably at least one other ingredient being present in the agent. The agents according to the invention can in particular contain surfactants, builders, polymers, glass corrosion inhibitors, corrosion inhibitors, bleaching agents, such as peroxygen compounds, bleach activators or bleach catalysts. They may also contain water-miscible organic solvents, further enzymes, enzyme stabilizers, sequestering agents, electrolytes, pH regulators and/or further auxiliaries, such as optical brighteners, graying inhibitors, dye transfer inhibitors, foam regulators, as well as dyes and fragrances, and combinations thereof. Advantageous ingredients of agents according to the invention are disclosed in international patent application WO 2009/121725, starting at the penultimate paragraph of page 5 and ending after the second paragraph on page 13. Reference is expressly made to this disclosure and the disclosure therein is incorporated into the present patent application.

An agent according to the invention advantageously contains the protease according to the invention in an amount of 2 μg to 20 mg, preferably of 5 μg to 17.5 mg, particularly preferably of 20 μg to 15 mg, and very particularly preferably of 50 μg to 10 mg per g of the agent. In various embodiments, the concentration of the protease (active enzyme) described herein in the agent is >0 to 1 wt. %, preferably 0.0001 or 0.001 to 0.1 wt. %, based on the total weight of the agent or composition.

An agent according to the invention contains the protease in an amount, increasingly preferably, of 1×10⁻⁸ to 5 wt. %, of 0.0001 to 1 wt. %, of 0.0005 to 0.5 wt. %, of 0.001 to 0.1 wt. %, in each case relative to active protein and relative to the total weight of the washing agent.

The embodiments of the present invention include all solid, powdered, liquid, gel or pasty administration forms of agents according to the invention, which may optionally also consist of a plurality of phases and can be present in compressed or uncompressed form. The agent can be present as a free-flowing powder, in particular having a bulk density of 300 g/l to 1200 g/l, in particular 500 g/l to 900 g/l or 600 g/l to 850 g/l. The solid administration forms of the agent further include extrudates, granules, tablets or pouches. Alternatively, the agent can also be in a liquid, gel or paste form, for example in the form of a non-aqueous liquid washing agent or a non-aqueous paste or in the form of an aqueous liquid washing agent or a water-containing paste. Liquid agents are generally preferred. Furthermore, the agent can be present as a single-component system. Such agents consist of one phase. Alternatively, an agent can also consist of a plurality of phases. Such an agent is accordingly divided into a plurality of components.

The proteases according to the invention are preferably used in liquid washing agents for cleaning textiles, particularly preferably in liquid washing agents having a pH of approximately 8 to approximately 9.

Washing or cleaning agents according to the invention can contain only one protease according to the invention. Alternatively, they can also contain further hydrolytic enzymes or other enzymes in a concentration expedient for the effectiveness of the agent. A further embodiment of the invention is thus represented by agents that further comprise one or more further enzymes. Further enzymes which can preferably be used are all enzymes which can exhibit catalytic activity in the agent according to the invention, in particular a lipase, amylase, cellulase, hemicellulase, mannanase, tannase, xylanase, xanthanase, xyloglucanase, β-glucosidase, pectinase, carrageenase, perhydrolase, oxidase, oxidoreductase or a protease which is different from the proteases according to the invention, as well as mixtures thereof. Additional enzymes are contained in the agent advantageously in an amount of from 1×10⁻⁸ to 5 wt. %, based on active protein. Increasingly preferably, each further enzyme is contained in agents according to the invention in an amount of from 1×10⁻⁷ to 3 wt. %, from 0.00001 to 1 wt. %, from 0.00005 to 0.5 wt. %, from 0.0001 to 0.1 wt. % and particularly preferably from 0.0001 to 0.05 wt. %, based on active protein. Particularly preferably, the enzymes exhibit synergistic cleaning performance with respect to particular dirt or stains, i.e., the enzymes contained in the agent assist one another in their cleaning performance. Very particularly preferably, such synergy exists between the protease present according to the invention and a further enzyme of an agent according to the invention. Synergistic effects can occur not only between different enzymes but also between one or more enzymes and other ingredients of the agent according to the invention.

Textile washing agents preferred according to the invention have at least one protease and at least one amylase. In a further preferred embodiment of the invention, textile washing agents have at least one protease and at least one cellulase. In a further preferred embodiment, textile washing agents have at least one protease, and at least one lipase. In a further preferred embodiment, textile washing agents have at least one protease, at least one amylase, and at least one lipase. In a further preferred embodiment, textile washing agents have at least one protease, at least one amylase, and at least one cellulase. In a further preferred embodiment, textile washing agents have at least one protease, at least one amylase, at least one cellulase, and at least one lipase. Textile washing agents which have 3 to 10 different enzymes are particularly preferred, it being possible for textile washing agents which have 3 to 10 different types of enzymes to be particularly preferred with regard to the cleaning performance over a very broad spectrum of stains.

Examples of proteases are the subtilisins BPN′ from Bacillus amyloliquefaciens and Carlsberg from Bacillus licheniformis, protease PB92, subtilisins 147 and 309, the protease from Bacillus lentus, subtilisin DY, and the enzymes thermitase, proteinase K and proteases TW3 and TW7, which in the narrower sense are associated with the subtilases but no longer with the subtilisins. Subtilisin Carlsberg is available in a developed form under the trade name Alcalase® from Novozymes. Subtilisins 147 and 309 are marketed by Novozymes under the trade names Esperase® and Savinase®, respectively. Protease variants, described, for example, in WO 95/23221, WO 92/21760 WO 2013/060621 and EP 3660151 are derived from the protease from Bacillus lentus DSM 5483. Other proteases that are suitable are, for example, the enzymes available under the trade names Durazym®, Relase®, Everlase®, Nafizym®, Natalase®, Kannase®, Progress Uno 101L® and Ovozyme® from Novozymes, the enzymes available under the trade names Purafect®, Purafect® OxP, Purafect® Prime, Excellase® and Properase®, Preferenz P100® and Preferenz P300® from Danisco/DuPont, the enzyme available under the trade name Lavergy pro 104 LS® from BASF, the enzyme available under the trade name Protosol® from Advanced Biochemicals Ltd., the enzyme available under the trade name Wuxi® from Wuxi Snyder Bioproducts Ltd., the enzymes available under the trade names Proleather® and Protease PR from Amano Pharmaceuticals Ltd., and the enzyme available under the name Proteinase K-16 from Kao Corp. The proteases from Bacillus gibsonii and Bacillus pumilus, which are disclosed in WO 2008/086916, WO 2007/131656, WO 2017/215925, WO 2021/175696 and WO 2021/175697, are particularly preferably used. Further proteases that can be used are those which are naturally present in the microorganisms Stenotrophomonas maltophilia, in particular Stenotrophomonas maltophilia K279a, Bacillus intermedius and Bacillus sphaericus.

Examples of amylases are the «-amylases from Bacillus licheniformis, Bacillus amyloliquefaciens or Bacillus stearothermophilus, as well as in particular the developments thereof that have been improved for use in washing or cleaning agents. The enzyme from Bacillus licheniformis is available from Novozymes under the name Termamyl® and from Danisco/DuPont under the name Purastar® ST. Development products of this «-amylase are available under the trade names Duramyl® and Termamyl® ultra (both from Novozymes), Purastar® OxAm (Danisco/DuPont) and Keistase® (Daiwa Seiko Inc.). The «-amylase from Bacillus amyloliquefaciens is marketed by Novozymes under the name BAN®, and derived variants from the α-amylase from Bacillus stearothermophilus are marketed under the names BSG® and Novamyl®, also by Novozymes. Others that are particularly noteworthy for this purpose are the «-amylases from Bacillus sp. A 7-7 (DSM 12368) and the cyclodextrin glucanotransferase (CGTase) from Bacillus agaradherens (DSM 9948) should be emphasized. Fusion products of all mentioned molecules can also be used. Furthermore, the developments of the «-amylase from Aspergillus niger and A. oryzae, available under the trade name Fungamyl® from Novozymes, are suitable. Other commercial products that can be advantageously used are, for example, Amylase-LT® and Stainzyme® or Stainzyme® ultra or Stainzyme® plus as well as Amplify™ 12L or Amplify Prime™ 100L, the latter also from Novozymes, and the PREFERENZ S® series from Danisco/DuPont, including, for example, PREFERENCE $100®, PREFERENCE S1000® or PREFERENCE S210®. Variants of these enzymes that can be obtained by point mutations may also be used according to the invention.

Suitable cellulases include those of bacterial or fungal origin. Chemically modified or protein-engineered mutants are included. Suitable cellulases are cellulases from the genera Bacillus, Pseudomonas, Humicola, Fusarium, Thielvia, Acremonium, e.g., the fungal cellulase from Humicola insolens, Mycelophthora thermophila and Fusarium oxysporum, which are disclosed in U.S. Pat. Nos. 4,435,307, 5,648,263, 5,691,178, 5,776,757 and WO 89/09259. Particularly suitable cellulases are the alkaline or neutral cellulases with color care properties. Examples of such cellulases are cellulases which are described in EP 0495257, EP 0531372, WO 96/11262, WO 96/29397 and WO 98/08940. Examples of cellulases with endo-1,4-glucanase activity (EC 3.2.1.4) are described in WO 2002/099091, for example those having a sequence of at least 97% identity to the amino acid sequence of positions 1 to 773 of SEQ ID NO:2 of WO 2002/099091. A further example can comprise a GH44-xyloglucanase, for example a xyloglucanase enzyme having a sequence of at least 60% identity to positions 40 to 559 of SEQ ID NO:2 of WO 2001/062903. Commercially available cellulases include Celluzyme™, Carezyme™, Carezyme Premium™, Celluclean™ (e.g., Celluclean™ 5000L and Celluclean™ 4000T), Celluclean Classic™, Cellusoft™, Endolase®, Renozyme® and Whitezyme™ (Novozymes A/S), Clazinase™ and Puradax HA™ (Genencor International Inc.), KAC-500 (B)™ (Kao Corporation), Revitalenz™ 1000, Revitalenz™ 2000 and Revitalenz™ 3000 (DuPont), as well as Ecostone® and Biotouch® (AB Enzymes).

Further enzymes that can be used are, for example, lipases or cutinases, in particular for the triglyceride-cleaving activities thereof, but also so as to create peroxy acids in situ from suitable precursors. Suitable lipases and cutinases are those of bacterial or fungal origin. Chemically modified mutated enzymes generated by protein engineering are included. Examples include lipase from Thermomyces, e.g., from T. Lanuginosus (formerly called Humicola lanuginosa), as described in EP 0258068 and EP 0305216, Humicola, e.g., H. insolens (WO 96/13580), lipase from strains of Pseudomonas (some of these now renamed Burkholderia), e. g, P. alcaligenes or P. pseudoalcaligenes, P. cephalia, P. sp. strain SD705, P. wisconensis, GDSL-type Streptomyces lipases, cutinase from Magnaporthegrisea, cutinase from Pseudomonas mendocina, lipase from Thermobida fusca, lipase from Geobacillus stearothermophilus, lipase from Bacillus subtilis and lipase from Streptomycesgriseus and S. pristinaespiris. The lipases that can originally be obtained from Humicola lanuginosa (Thermomyces lanuginosus) or have been developed therefrom, in particular those having one or more of the following amino acid exchanges in positions D96L, T213R and/or N233R, particularly preferably T213R and N233R, proceeding from the mentioned lipase, belong to the preferred lipases. Preferred commercial lipase products include Lipolase™, Lipex™, Lipolex™, and Lipoclean™ (Novozymes A/S), Lumafast (Genencor/DuPont), and Lipomax (Gist-Brocades).

In order to increase the bleaching effect, oxidoreductases, such as oxidases, oxygenases, catalases, peroxidases, such as halo, chloro, bromo, lignin, glucose, or manganese peroxidases, dioxygenases or laccases (phenoloxidases, polyphenoloxidases), can be used according to the invention. Advantageously, organic, particularly preferably aromatic compounds that interact with the enzymes are additionally added in order to potentiate the activity of the relevant oxidoreductases (enhancers) or, in the event of greatly differing redox potentials, to ensure the flow of electrons between the oxidizing enzymes and the stains (mediators).

In the cleaning agents described herein, the enzymes to be used can further be formulated together with accompanying substances, for example from fermentation. In liquid formulations, the enzymes are preferably used as liquid enzyme formulation(s).

The enzymes are generally not provided in the form of the pure protein, but rather in the form of stabilized, storable and transportable preparations. These ready-made preparations include, for example, the solid preparations obtained by means of granulation, extrusion or lyophilization or, in particular in the case of liquid or gel agents, solutions of the enzymes, which are advantageously as concentrated as possible, have a low water content, and/or are admixed with stabilizers or further auxiliaries.

Alternatively, the enzymes can be encapsulated both for the solid and for the liquid administration form, for example by means of spray-drying or extrusion of the enzyme solution together with a preferably natural polymer or in the form of capsules, for example those in which the enzymes are enclosed as in a solidified gel, or in those of the core-shell type, in which an enzyme-containing core is coated with a protective layer that is impermeable to water, air and/or chemicals. Further active ingredients, for example, stabilizers, emulsifiers, pigments, bleaches or dyes can additionally be applied in overlaid layers. Such capsules are made using methods that are known per se, for example by means of vibratory granulation or roll granulation or by means of fluid bed processes. Advantageously, such granules are low in dust, for example due to the application of polymeric film formers, and are stable in storage due to the coating.

Furthermore, it is possible to formulate two or more enzymes together such that a single granule exhibits a plurality of enzyme activities.

The enzymes can also be introduced into water-soluble films, such as those used in the formulation of washing and cleaning agents in a unit dosage form. Such a film allows the enzymes to be released after contact with water. As used herein, “water-soluble” refers to a film structure that is preferably completely water-soluble. Preferably, such a film consists of (completely or partially hydrolyzed) polyvinyl alcohol (PVA).

A further object of the invention is a method for cleaning textiles and/or hard surfaces, in particular dishes, which is characterized in that in at least one method step, an agent according to the invention is used. In various embodiments, the method described is characterized in that the tannase is used at a temperature of approximately 0° C. to approximately 100° C., preferably approximately 20° C. to approximately 60° C. and more preferably approximately 20° C. to approximately 40° C. 20° C. represents a low washing temperature, while 40° C. is the preferred/average washing temperature in Europe.

This includes both manual and machine methods, with machine methods being preferred because they can be controlled more precisely, for example with regard to the quantities used and contact times. Methods for cleaning textiles are generally characterized by the fact that, in a plurality of method steps, various cleaning-active substances are applied to the material to be cleaned and washed off after the exposure time, or in that the material to be cleaned is otherwise treated with a washing agent or a solution or dilution of this agent.

Because proteases according to the invention by nature already have hydrolytic activity and also exhibit said activity in media that otherwise have no cleaning power, such as, for example, in a simple buffer, an individual and/or the single step of such a method can consist of bringing a protease according to the invention into contact with the stain as the only cleaning component, preferably in a buffer solution or in water. This represents a further embodiment of this subject matter of the invention.

Alternative embodiments of this subject matter of the invention also include methods for treating textile raw materials or for textile care, in which a protease according to the invention is active in at least one method step. Among these, methods for textile raw materials, fibers or textiles comprising natural constituents are preferred, and very particularly for those comprising wool or silk.

Finally, the invention also covers the use of the proteases described herein in washing or cleaning agents, for example as described above, for the (improved) removal of protease-sensitive stains, which are preferably selected from the group consisting of blood, egg (yolk), milk and other protein-containing stains.

In a further preferred embodiment, the invention relates to the use of a protease according to the invention as described herein in a washing or cleaning agent for improving the cleaning performance of such a protease-containing washing or cleaning agent, in particular of a liquid washing or cleaning agent, on at least one and increasingly preferably on two, three, four, five or six protease-sensitive stains(s), which is/are preferably selected from the group consisting of blood, egg (yolk), milk and other protein-containing stains, wherein the improvement in the cleaning performance of an agent containing a protease according to the invention relative to an agent without protease and/or relative to an agent containing a reference protease is determined as described in example 2, in particular in a temperature range of about 20° C. to about 40° C.

In a further preferred embodiment, the invention relates to the use of a protease according to the invention as described herein in a washing or cleaning agent for improving the cleaning performance of such a protease-containing washing or cleaning agent, in particular of a liquid washing or cleaning agent, on at least one and increasingly preferably on two, three, four, five or six protease-sensitive stains(s), which is/are preferably selected from the group consisting of blood, egg (yolk), milk and other protein-containing stains, wherein the improvement in the cleaning performance of an agent containing a protease according to the invention relative to an agent without protease and/or relative to an agent containing a reference protease is determined as described in example 2, in particular in a temperature range of about 20° C. to about 40° C., wherein the protease is a protease which has proteolytic activity and is 75% and increasingly preferably 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9% or 100% identical.

All elements, subjects and embodiments that are described for the protease according to the invention and agents that contain them can also be applied to this subject of the invention. Therefore, reference is expressly made at this point to the disclosure at the corresponding point where it is indicated that this disclosure also applies to the methods and uses according to the invention.

### EXAMPLES

TABLE 1
WASHING AGENT MATRIX USED

| | wt. % of active substance | wt. % of active substance |
|---|---|---|
| Chemical name | in the raw material | in the formulation |
| Demineralized water | 100% | Remainder |
| LAS | 96% | 3-20% |
| FAEOS | 70% | 3-8% |
| Palm kernel oleic acid | 30% | 0.3-4% |
| FAEO | 100% | 2-11% |
| HEDP | 60% | 0.5-<2% |
| Citric acid | 100% | 1-5% |
| NaOH | 50% | 0.5-2% |
| Defoamer | 100% | <1% |
| Glycerol | 99.5% | 1-3% |
| 1,2-propanediol | 100% | 8-12% |
| Monoethanolamine | 100% | 4-8% |
| Soil repellent polymer | 30% | 0.1-1% |
| Stabilizer | 100% | 0.1-0.5% |
| Perfumes, DTI | t.q. | minors |

Dosage 3.17 g/L;
pH 8.2 to 8.4

### Example 1: Determining the Protease Activity

Protease activity was determined in a discontinuous assay using casein as substrate. The final concentration of the substrate solution was 12 mg/ml casein (prepared according to Hammarsten; Merck, Darmstadt, #2242) and 30 mM Tris in synthetic tap water. Synthetic tap water is a solution of 0.029% (w/v) CaCl₂)·2H₂O, 0.014% (w/v) MgCl₂·6 H₂O and 0.021% (w/v) NaHCO₃ with 15° dH (German hardness). The substrate solution was heated to 70° C. and the pH was adjusted to 8.5 at 50° C. using 0.1 N NaOH. The protease solution was prepared by adding 2% (w/v) water-free pentasodium tripolyphosphate to synthetic tap water and adjusting to pH 8.5 with hydrochloric acid. 200 μl of the enzyme solution were added to 600 μl of the casein solution. The mixture was incubated at 50° C. for 15 minutes. The reaction was terminated by addition of 600 μl of 0.44 M trichloroacetic acid (TCA), 0.22 M sodium acetate in 3% (w/v). After a cooling step of 15 minutes on ice, the TCA insoluble protein was removed by centrifugation. 900 μl of the remaining solution was mixed with 300 μl of 2 N NaOH and the absorption of this mixture containing TCA-soluble proteins was measured at 290 nm. Control values were generated by adding 600 μl TCA solution to 600 μl casein solution, followed by addition of 200 μl enzyme solution. A protease solution which causes an absorption change of 0.50 OD at 290 nm under these conditions has, according to the present designation, an activity of 10 HPE per ml (cf. for example Schülein et al., Mol Gen Gent (1991) 227:137-143).

### Example 2: Determining the Cleaning Performance

### Mini Wash Test

The cleaning performance was determined in mini-wash tests with Bacillus subtilis culture supernatants containing the expressed protease according to SEQ ID NO:1. The supernatants were used with the same activity as the benchmark (6 HPE/ml active enzyme protein in the washing liquor).
Conditions: 20° C. and 40° C., 16° dH water, 1 h

### Stains:

1. WFK 10 N (whole egg/soot)
2. CFT C-05 (blood/milk/ink)
3. CFT C-03 (cocoa/soot)

Punched out woven fabric (diameter=10 mm) was placed in microtiter plates, the washing liquor was adjusted to pH=8 and preheated to the relevant target temperature (20° C. or 40° C.), final concentration 3.17 g/L. Washing liquor without enzymes and wash liquor with protease supernatant were applied to the stain and incubated for 60 min at 40° C. and 600 rpm or for 60 min at 20° C. and 600 rpm. The stains were then rinsed repeatedly with clear water, dried and the brightness was determined using a colorimeter (MACH 5, Multi area color-measurement, CFTBV.nl). The lighter the fabric, the better the cleaning performance. The Y value=brightness is measured here, the higher the brighter. The absolute values of the brightness measurements of the test textiles after washing in washing liquor without protease are approximately between Y=10-50. Tables 2 and 3 show the ΔY values, i.e. the difference between the respective test batches (=washing agent with protease) and the control (=washing agent without protease). A difference greater than 0.5 is considered to be significant in the method used.

TABLE 2
WASHING PERFORMANCE AT 20° C.

| | Savinase | Protease SEQ ID |
|---|---|---|
| | 16L | NO:1 |
| 10 N | 2.88 | 3.44 |
| C-05 | 0.68 | 1.6 |
| C-03 | 1.64 | 1.6 |
| Overall | 5.2 | 6.64 |
| performance | | |

TABLE 3
WASHING PERFORMANCE AT 40° C.

| | Savinase | Protease SEQ ID |
|---|---|---|
| | 16L | NO:1 |
| 10 N | 0.22 | 2.17 |
| C-05 | 1.62 | 1.39 |
| C-03 | 1.56 | 2.62 |
| Overall | 3.4 | 6.18 |
| performance | | |

The protease according to the invention shows comparable or significantly improved washing performance on different stains compared to a conventional protease. This is particularly remarkable because the protease according to SEQ ID NO:1 is a newly isolated wild-type enzyme that has not yet been modified by protein engineering, and yet shows comparable or improved cleaning performance relative to commercially available proteases for washing and cleaning agents.

### Example 3: Example Formulations

TABLE 4
LIQUID WASHING AGENTS

| | wt. % of | | | | | |
|---|---|---|---|---|---|---|
| | active | | | | | |
| | substance | | | | | |
| | in the raw | Wt. % of active substance in the formulation | | | | |
| Chemical name | material | A | B | C | D | E |
| Demineralized water | 100% | Remainder | Remainder | Remainder | Remainder | Remainder |
| LAS | 96% | 5.5% | 20% | 15.0% | 5.5% | 21.7% |
| FAEOS | 70% | 7.0% | | 5.0% | | |
| Palm kernel oleic acid | 30% | 3.0% | 8.0% | | | 7.0% |
| FAEO | 100% | 5.5% | | 8.0% | | |
| C13/15 oxo alcohol, 8 EO | | | 25% | | | |
| C12-18 fatty alcohol ethoxylate, | | | | | | 22.4% |
| 7 EO | | | | | | |
| Alkyl polyglycoside | | | | 4.0% | | |
| Non-ionic surfactants | | | | | 3.1% | |
| Soap | | | | 1.0% | 0.5% | |
| HEDP | 60% | 0.5% | | | | |
| DTPMPA 7Na | | | 1.0% | 1.0% | 0.2% | 0.5% |
| Citric acid | 100% | 2.5% | | 3.0% | 0.23% | |
| NaOH | 50% | 3.0% | | | 0.7% | |
| Glycerol | 99.5% | 3.0% | 5.0% | | 0.5% | 9.4% |
| Ethanol | | 1.5% | 3.0% | | | |
| 1,2-propanediol | 100% | | 10.0% | 12.0% | | 5.0% |
| Monoethanolamine | 100% | | 6.0% | 7.0% | | 6.0% |
| Boric acid | 100% | 1.0% | | 1.0% | 0.5% | |
| Soil release polymer | | | 1.5% | 2.0% | | |
| Polyalkoxylated alkanolamine | t.q. | | | | | 4.5% |
| Ethoxylated polyethyleneimine | t.q. | | | | | 4.5% |
| Protease SEQ ID NO: 1 | t.q. | 6 HPE/ml | 6 HPE/ml | 6 HPE/ml | 6 HPE/ml | 6 HPE/ml |
| Fragrance, DTI, other enzymes, | t.q. | minors | minors | minors | minors | minors |
| defoamers, etc. | | | | | | |
| Dosage 3.17 g/L; pH 8.0-8.4 | | | | | | |

TABLE 5
HAND DISHWASHING AGENT

| | wt. % of active | wt. % of active substance |
|---|---|---|
| | substance | in |
| Chemical name | in the raw material | the formulation |
| Demineralized water | 100% | Remainder |
| FAEOS | | 8.8% |
| Cocoamidopropyl | | 1.2% |
| betaine | | |
| Calcium lactate | | 1.0% |
| Amylase (Stainzyme | t.q. | 0.8% |
| 12L) | | |
| Perfume | t.q. | 0.2% |
| Dye | t.q. | 0.01% |
| Salts | | 2.0% |
| Protease SEQ ID NO:1 | t.q. | 6 HPE/ml |
| pH | | 8.0 |

1. A protease, comprising:
an amino acid sequence which has at least 82%, sequence identity to the amino acid sequence given in SEQ ID NO: 1 over its entire length.

2. The protease of claim 1, wherein:
(a) the protease is obtained from a protease according to claim 1 as a starting molecule by single or multiple conservative amino acid substitution(s); and/or
(b) the protease is obtained from a protease according to claim 1 as a starting molecule by means of fragmentation or deletion, insertion or substitution mutagenesis, and further comprises an amino acid sequence which matches the starting molecule over a length of at least 190, 200, 210, 220, 230, 240, 250, 260, 270, 271, 272, 273, 274 or 275 contiguous amino acids.

3. The protease according to claim 1, wherein the protease causes improved cleaning performance of a washing or cleaning agent containing a protease according to the invention, relative to an agent without protease and/or relative to an agent containing a reference protease, on at least one protease-sensitive stain, which is selected from the group consisting of: blood, egg yolk, milk and other protein-containing stains, wherein the cleaning performance is determined as described in example 2.

4. A method for producing a protease according to claim 1, comprising:
providing a starting protease having at least 75% sequence identity to the amino acid sequence given in SEQ ID NO:1 over its entire length.

5. The method according to claim 4, further comprising one or more of the following method steps:
(a) introducing single or multiple conservative amino acid substitution(s) into the starting protease;
(b) altering the amino acid sequence of the starting protease by fragmentation or deletion, insertion or substitution mutagenesis such that the protease comprises an amino acid sequence that matches the starting tannase over a length of at least 190, 200, 210, 220, 230, 240, 250, 260, 270, 271, 272, 273, 274 or 275 contiguous amino acids.

6. A nucleic acid coding for a protease according to claim 1.

7. A vector containing a nucleic acid according to claim 6.

8. A non-human host cell that contains at least one selected from the group of: a nucleic acid, or a vector, encoding a protease according to claim 1; and, wherein a protease of claim 1 is secreted into the medium surrounding the host cell.

9. A method for producing a protease, comprising
a) cultivating a host cell according to claim 8 and
b) isolating the protease from the culture medium or from the host cell.

10. A washing or cleaning agent, comprising:
at least one protease according to claim 1;
wherein the concentration of the at least one protease is from 0.00005 to 15 wt. %, based on active protein, and/or wherein the agent is substantially free from boron-containing compounds, and/or wherein the agent has a pH of about 8 to about 9, and/or wherein the agent is substantially free from phosphonate-containing compounds, and/or wherein the agent is substantially free from phosphate-containing compounds.

11. A method for cleaning textiles or hard surfaces, characterized in that an agent according to claim 10 is used in at least one method step.

12. The protease of claim 1 wherein the protease is effective for removing protease-sensitive stains selected from the group consisting of: blood, egg yolk, milk, and other protein-containing stains.

13. The protease of claim 1, wherein the protease is in a washing or cleaning agent, and wherein the washing or cleaning agent exhibits improved performance on at least one protease-sensitive stain selected from the group consisting of; blood, egg yolk, milk, and other protein-containing stains, wherein the improvement in the cleaning performance of the washing or cleaning agent is determined relative to a washing or cleaning agent without protease and/or relative to a washing or cleaning agent containing a reference protease as described in example 2, at a temperature range of about 20° C. to about 40° C.

14. A nucleic acid coding for a protease according to claim 1.
